(19) United States
(12) Patent Application Publication
(10) Pub. No.: US 2012/0109275 A1
(43) Pub. Date: May 3, 2012

(54) STENT WITH RADIALLY ASYMMETRIC FORCE DISTRIBUTION

(75) Inventor: Jens Ulmer, Zuerich (CH)

(73) Assignee: BIOTRONIK AG, Buelach (CH)

(21) Appl. No.: 13/272,559

(22) Filed: Oct. 13, 2011

Related U.S. Application Data
(60) Provisional application No. 61/407,922, filed on Oct. 29, 2010.

(51) Int. Cl. A61F 2/84; A61F 2/82

(52) U.S. Cl. 623/1.11; 623/1.15; 623/1.16

(57) ABSTRACT

An expandable stent comprising a tubular main body with a lumen along a longitudinal axis, characterized in that the main body has at least one radially asymmetrically expandable portion, wherein the radially asymmetrically expandable portion has an expandability which is increased in comparison to the average expandability in the rest of the main body of the stent.

### CROSS REFERENCE TO RELATED APPLICATIONS

This invention claims benefit of priority to U.S. patent application Ser. No. 61/407,922, filed Oct. 29, 2010; the contents of which are herein incorporated by reference in their entirety.

### TECHNICAL FIELD

The invention relates to an expandable stent and to a catheter for applying a stent.

### BACKGROUND

The implantation of stents has become established as one of the most effective therapeutic measures in the treatment of vascular diseases. Stents have the purpose of assuming a support function in the hollow organs of a patient. To this end, stents of the conventional type comprise a main body which has a plurality of circumferential support structures, e.g. consisting of metal struts, which are initially in a compressed form for the purpose of insertion into the body and are expanded at the site of application. One of the main fields of application of such stents is to permanently or temporarily widen and keep open vascular constrictions, in particular constrictions (stenoses) of the coronary vessels. In addition, aneurysm stents are also known for example, which are used to support damaged vessel walls.

Stents have a tubular main body which includes a lumen along a longitudinal axis. The main body has a plurality of circumferential support structures, e.g. circumferential cylindrical meander-shaped rings or helices, which are arranged one after the other along the longitudinal axis. These support structures are each composed of a sequence of diagonal elements and arched elements (also referred to as crowns) and form the geometric basic unit of modern stent designs. The support structures are connected to one another by connecting elements—so-called connectors—in the longitudinal direction. On the one hand these connectors must be arranged in such a way that they ensure a sufficient bending flexibility of the stent, but on the other hand they must not hinder a crimping and/or dilation process.

The stents of the prior art are characterized in that they have an axisymmetric radial force distribution during expansion. As a result, the radial force exerted on the vessel walls is substantially evenly distributed around the circumference of the stent. However, most stenoses to be treated do not exhibit a radially symmetrical size or strength profile within the vessel (so-called eccentric stenosis). As a result, when such eccentric stenoses are treated using commercially available stents, surrounding healthy tissue is unnecessarily to subjected to stress and injuries may occur to the vessel wall during the treatment. Such traumatological changes to the vessel wall, including and specifically in healthy tissue, are nowadays considered to be a main trigger for the formation of new tissue (neointimal hyperplasia) and thus to be associated with the risk of restenosis.

### SUMMARY

The object of the present invention is to alleviate or avoid one or more disadvantages of the prior art. In particular, one object of the present invention is to provide means which allow the treatment of stenoses but which subject surrounding healthy tissue to less stress.

The present invention achieves this object by providing an expandable stent comprising a tubular main body with a lumen along a longitudinal axis, characterized in that the main body has at least one radially asymmetrically expandable portion, wherein the radially asymmetrically expandable portion has an average expandability which is increased in comparison to the average expandability in the rest of the main body of the stent.

Preferably, the ratio δ of the average expandability in the rest of the main body of the stent to the average expandability in the radially asymmetrically expandable portion is 1>δ≧0.01, preferably 0.95≧δ≧0.1 and particularly preferably 0.9≧δ≧0.7.

The stent according to the invention is characterized in that the radial forces acting on the vessel wall are distributed asymmetrically during the expansion. During the expansion, regions with an increased expandability expand to a greater degree than the other regions of the main body, which require the application of an increased force in order to expand (lower expandability). As a result, by applying a uniform radially symmetrical force (for example in the form of the inflation pressure through the dilation balloon), the stent expands to a greater extent in regions with increased expandability than in regions with a s lower expandability. The stent according to the invention makes it possible to treat eccentric constrictions of vessels in a particularly advantageous manner, wherein the diseased regions of the vessel wall are preferentially widened due to the increased expandability of the asymmetrically dilatable stent, while the tissue in the surrounding healthy parts of the vessel wall is not expanded to such a great extent. A stenosis can thus effectively be eliminated and the blood flow can be restored, largely without damaging the surrounding healthy vascular tissue. By using the stent according to the invention, the risk of undesirable new tissue formation (neointimal hyperplasia) is reduced without the use of medicaments or active substances, and thus the likelihood of restenosis occurring is accordingly reduced.

The expandable stent according to the invention has a tubular main body which encloses a lumen along a longitudinal axis. Following application of the stent in a blood vessel, blood is able to flow through this lumen. The main body comprises a plurality of circumferential support structures which are arranged one after the other along the longitudinal axis and enclose the lumen.

The support structures are each composed of a sequence of diagonal elements and arched elements. The diagonal elements have an elongated shape with two ends and connect two arched elements having oppositely directed curvatures. The diagonal elements are largely responsible for the extension of the support structure in the direction of the longitudinal axis. The arched elements are curved and connect two successive diagonal elements of a support structure to one another in such a way that these come to lie one above the other along an axis running vertically with respect to the longitudinal axis, resulting in an annular circumferential structure which encloses a lumen.

In addition to a plurality of support structures, the main body comprises one or more connectors, wherein two successive circumferential support structures are connected to one another via at least one connector. The connectors of the stent according to the invention are preferably configured in such a way that a plurality of support structures can be connected in order to form a main body which is suitable for use in an expandable stent. To this end, in each case one connector is connected at a first end to a diagonal element or an arched element of a first support structure and at a second end to a diagonal element or an arched element of a second support structure. Two successive support structures may also be connected to one another via more than one connector. One, several or all connectors of the stent according to the invention may have a substantially elongated shape with two opposite ends. Preferably, the connectors are long enough to ensure a sufficient flexibility of the two adjacent support structures, but not so long that the stent according to the invention becomes torsionally flexible. One, several or all connectors of the stent according to the invention may have a more or less curved shape. The connectors are oriented in principle in the longitudinal direction between the two circumferential support structures to be connected, the connectors not necessarily being aligned parallel to the longitudinal axis. In the expanded state of the stent, the connectors may form an angle is relative to the longitudinal axis.

The main body of the stent according to the invention may be made from any implant material which is suitable for the manufacture of implants, in particular stents. Implant materials for stents include polymers, metallic materials and ceramic materials. Biocompatible metals and metal alloys for permanent implants include for example stainless steels (for example 316L), cobalt base alloys (for example CoCrMo casting alloys, CoCrMo forging alloys, CoCrWNi forging alloys and CoCrNiMo forging alloys), pure titanium and titanium alloys (e.g. cp titanium, TiAl6V4 or TiAl6Nb7) and gold alloys. Preferably, the main body comprises or consists of a metallic implant material.

With particular preference, the stent according to the invention has a main body which contains or consists of a biodegradable implant material. In the field of biocorrodible stents, it is proposed to use magnesium or pure iron, or biocorrodible base alloys of the elements magnesium, iron, zinc, molybdenum and tungsten. In particular, the main body of a stent according to the invention may contain or consist of a biocorrodible magnesium alloy.

In the present case, alloy is understood to mean a metallic material having magnesium, iron, zinc or tungsten as the main component. The main component is the alloy component having the highest proportion by weight in the alloy. A proportion of the main component is preferably more than 50% by weight, in particular more than 70% by weight.

The alloys of the elements magnesium, iron, zinc or tungsten are to be selected in terms of their composition such that they are biocorrodible. In the context of the invention, biocorrodible is used to denote alloys in which, in a physiological environment, a degradation takes place which ultimately leads to the entire implant or the part of the o implant formed from the material losing its mechanical integrity. As the test medium for testing the corrosion behavior of an alloy in question, use is made of artificial plasma as specified according to EN ISO 10993-15:2000 for biocorrosion testing (composition: NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). To this end, a sample of the alloy to be tested is stored together with a defined quantity of the test medium in a sealed sample container at 37° C. The samples are removed at time intervals—tailored to the expected corrosion behavior—from a few hours to several months and are analyzed in a known manner for signs of corrosion. The artificial plasma according to EN ISO 10993-15:2000 corresponds to a medium that is similar to blood and thus provides a possibility of duplicating in a reproducible manner a physiological environment within the meaning of the invention.

DE 197 31 021 A1 discloses suitable biocorrodible metallic implant materials having as the main component an element from the group consisting of alkali metals, alkaline earth metals, iron, zinc and aluminum. Alloys based on magnesium, iron and zinc are described as being particularly suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc and iron. DE 102 53 634 A1 also discloses the use of a biocorrodible magnesium alloy having a proportion of magnesium >90%, yttrium 3.7-5.5%, rare earth metals 1.5-4.4% and others <1%, which is suitable in particular for producing an endoprosthesis, e.g. in the form of a self-expanding stent or a stent that can be expanded by means of a balloon.

The main body of the stent according to the invention has at least one radially asymmetrically expandable portion. A portion is understood here to mean a wall region of the main body which has both a dimension in the longitudinal direction and in the radial direction. A portion is radially asymmetrically expandable within the meaning of the present invention when this portion is preferentially expanded during the expansion compared to regions of the main body adjacent to the portion, and is thus suitable during the expansion for exerting a greater force on surfaces surrounding the main body than the regions of the main body adjacent to the portion.

To this end, the radially asymmetrically expandable portion of the stent has an average expandability which is increased in comparison to the average expandability in the rest of the main body, in particular in the regions of the main body adjacent to the radially asymmetrically expandable portion. The term expandability is understood to mean the property of a material to change its shape under the effect of force. The expandability indicates the extent to which a body, a material or a material composition can be extended without breaking or tearing. The person skilled in the art knows means and ways of determining the average expandability of certain portions or regions of the main body of the stent according to the invention. Since the invention concerns a relative ratio of the average expandabilities relative to one another, the person skilled in the art can freely choose the method, provided that the average expandability in the radially asymmetrically expandable portion is measured using the same method as the average expandability in the rest of the main body of the stent. Preferably, the ratio S of the average expandability in the rest of the main body of the stent to the average expandability in the radially asymmetrically expandable portion is 1>δ≧0.01, preferably 0.95≧δ≧0.1 and particularly preferably 0.9≧δ≧0.7.

It should be noted here that the expandability of the asymmetrically expandable portion should decrease as the expansion progresses (this is the case for example with the abovementioned CoCr alloys) and the material hardens. This prevents any tearing of the struts in regions of increased expandability during the full dilation.

The expandability may be influenced by a number of measures. By way of example, the expandability of a portion of the main body may be increased as a result of the fact that the arched elements in the radially asymmetrically expandable portion have an average strut width which is smaller than the average strut width of the arched elements in the rest of the main body. Given the same applied force, arched elements made from the same material and having the same geometry but with a smaller strut width expand to a greater extent than arched elements with a larger strut width. Preferably, the ratio φ of the average strut width of the arched elements in the radially asymmetrically expandable portion to the average strut width of the arched elements in the rest of the main body of the stent is 1>φ≧0.01, preferably 0.95≧φ≧0.1 and particularly preferably 0.9≧φ≧0.7.

As an alternative or in addition to varying the strut width of the arched elements, an increased expandability may be achieved by the fact that the average mesh width in the radially asymmetrically expandable portion is increased in comparison to the average mesh width in the rest of the main body. A change in the mesh width can be achieved for example by changing the geometry of connectors, arched elements and/or diagonal elements. Given the same applied force, regions of the main body made from the same material but with a larger mesh width expand to a greater extent than regions of the main body of the stent according to the invention having a smaller mesh width. Preferably, the ratio κ of the average mesh width in the rest of the main body of the stent to the average mesh width in the radially asymmetrically expandable portion is 1>κ≧0.01, preferably 0.95≧κ≧0.1 and particularly preferably 0.9≧κ≧0.7.

As an alternative or in addition, an increased expandability in the stent according to the invention may be achieved by the fact that the average spacing between the successive circumferential support structures in the radially asymmetrically expandable portion is greater than the average spacing between the successive circumferential support structures in the rest of the main body of the stent. A greater spacing between the support structures may be achieved for example by influencing the geometry of the connectors. Preferably, this can be achieved in that the average length of the connectors in the radially asymmetrically expandable portion is greater than the average length of the connectors in the rest of the main body of the stent according to the invention. Given the same applied force, regions of the main body made from the same material and with the same geometry but with a greater spacing between the successive support structures expand to a greater extent than regions of the main body of the stent according to the invention having a smaller spacing between the support structures. Preferably, the ratio c of the average spacing between the successive circumferential support structures in the rest of the main body of the stent to the average spacing between the successive circumferential support structures in the radially asymmetrically expandable portion is 1>ε≧0.01, preferably 0.95 ≧ε≧0.1 and particularly preferably 0.9≧ε≧0.7.

The shape, positioning and number of radially asymmetrically dilatable portions of the stent according to the invention can in principle be selected freely and may depend for example substantially on the shape, position and size of the area to be treated in the vessel.

The stent according to the invention may have for example just one single radially asymmetrically dilatable portion.

The stent according to the invention may also have a number of separate, successive or is overlapping radially asymmetrically dilatable portions.

The radially asymmetrically dilatable portions may be configured in a certain geometric shape. They may for example be, independently of one another, circular, elliptical or strip-shaped.

The stent according to the invention may be characterized for example in that it has a single radially asymmetrically dilatable portion and this extends substantially over the entire expandable main body.

If the stent according to the invention has more than one radially asymmetrically dilatable portion, this plurality may be configured and arranged in such a way that the sum of the longitudinal sizes of the individual radially asymmetrically dilatable portions accounts for no more than 50% of the longitudinal size of the expandable main body of the stent.

Given a suitable configuration and positioning, the presence of a plurality of radially asymmetrically dilatable portions on a stent according to the invention may for example also serve to prevent an undesired change in position of the stent during the expansion. In particular, the radially asymmetrically dilatable portions present in addition to the radially asymmetrically dilatable portions used for therapeutic purposes may be oriented and configured in such a way that an undesired rotational movement of the stent during the expansion in the vessel is avoided or is at least made more difficult. Such a change in position may occur for example when the radially asymmetrically dilatable portion meets the lesion to be expanded and is deflected by the latter or slips off the latter before the rest of the stent has been sufficiently widened to ensure a stable position in the vessel and thus to prevent any undesired rotational movement in the vessel.

The present invention also relates to a catheter for applying the stent according to the invention, the stent according to the invention being crimped onto the catheter. Suitable catheters are known to the person skilled in the art.

In order to make particularly effective use of the advantages of the stent according to the invention, it is useful to ensure that the stent, prior to expansion, is radially oriented at the desired location in the vessel in such a way that the radially asymmetrically expandable portion also comes to lie on the site that is to be treated, and not on surrounding healthy tissue.

For the success of the asymmetric force distribution by the stent according to the invention, it is therefore desirable that the stent can be precisely positioned within the vessel. Not only does the longitudinal position have to be ensured, but also the stent must be brought into the correct radial orientation by rotation.

To this end, the catheter according to the invention may be provided with means or markers which allow a precise positioning of the catheter within a vessel. It is known to the person skilled in the art that, besides the method described below, further methods and imaging processes exist for achieving the precise positioning of a catheter within the vessel. For example, using intravascular imaging processes (ultrasound, optical coherence tomography) in combination with a catheter, the latter can be precisely oriented within the vessel. Methods also exist for determining the spatial position of a catheter within a lumen with the aid of permanent magnets in the catheter tip.

For a simpler method, the following principle can be applied:

The surface of the inner shaft and/or outer shaft of the catheter may have one or more markers which are arranged and oriented in such a way that, during the application of the catheter in the vessel of the individual to be treated, the spatial orientation of the radially asymmetrically dilatable portion of the stent according to the invention can be determined. Preferably, the markers are positioned and grouped on the catheter in such a way that they are not covered by the crimped stent according to the invention and the rotational position of the radially asymmetrically dilatable portion of the stent in the vessel can be determined.

In this case, use is preferably made of markers which can be detected and displayed from to outside the body by means of imaging processes. Suitable markers are known to the person skilled in the art. These may be for example known X-ray and/or fluorescent markers. In order to allow a reliable detection and display of the three-dimensional position of the catheter in the vessel, preferably more than one marker substance or more than one marker is used, particularly preferably at least two different markers, very particularly preferably at least three different markers. By using a number of different markers, the risk of artifacts in the imaging is reduced.

The markers are preferably arranged in different geometric shapes on the surface of the inner shaft and/or outer shaft of the catheter. The geometric shapes are preferably rings, strips, triangles and/or arrows, the first-mentioned serving as reference systems for the subsequent image processing. These geometric shapes may be configured in such a way that, despite a two-dimensional imaging (in a type of “plan view”), the orientation of the catheter in three-dimensional space can be determined. To this end, different shapes may be combined with one another. The use of triangles, which may extend in particular around half the circumference of the catheter, is particularly advantageous. In one preferred embodiment, at least three identical or different geometric shapes are applied to and oriented on the surface of the catheter in such a way that it is possible to determine the rotational position of the radially asymmetrically dilatable portion(s) of the stent according to the invention that is crimped onto said catheter.

Preference is given to using at least two triangles, the tips of which are arranged in opposite directions. With particular preference, use is made of three triangles, the tips of which are alternately arranged in opposite directions to one another and the base of which is in each case arranged relative to one another in such a way that a clear two-dimensional image that is specific to each rotational position of the catheter is obtained. This image can be analyzed automatically using image analysis processes and can be converted directly into a rotational position. For this, the radio markers at the ends of the balloon are used as reference systems which make it possible to determine clearly both the surface area and the geometric shape (in the two-dimensional display thereof) of the triangular position markings.

### DESCRIPTION OF THE DRAWINGS

Figures:

FIGS. 1A-D show a schematic diagram of part of a main body of a stent, wherein a stent according to the prior art is shown in 1A, while 1B, 1C and 1D each show different embodiments of a stent according to the invention with a radially asymmetrically expandable portion.

FIGS. 2A-C show a schematic diagram of the configuration of radially asymmetrically dilatable portions of a stent according to the invention. 1A shows a diagram of a radially asymmetrically dilatable portion in the shape of a strip; 1B shows a diagram of a radially asymmetrically dilatable portion in the shape of an ellipse; and 1C shows a diagram of part of a stent with two elliptical radially asymmetrically dilatable portions.

FIG. 3 shows a two-dimensional diagram (as a “plan view”) of a catheter belonging to the stent according to the invention with markers arranged on the surface, in different stages of rotation.

### DETAILED DESCRIPTION

The invention will be explained in more detail below with reference to exemplary embodiments.

### Exemplary Embodiment 1

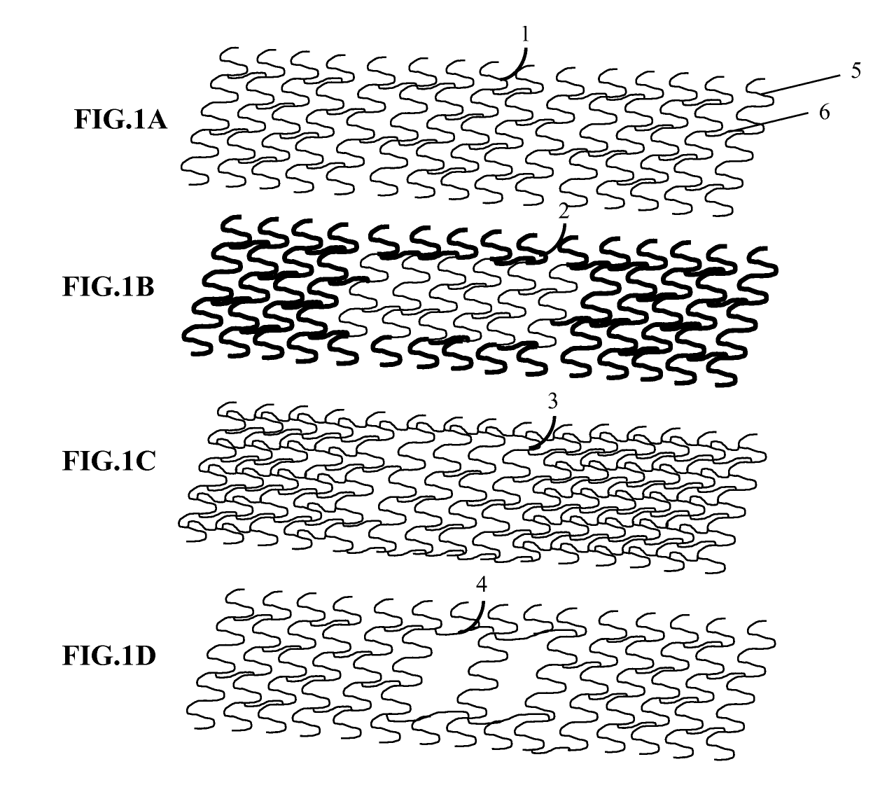

FIG. 1A shows a region of a main body of a conventional stent. It is shown in a part 1 that the main body is formed of a regular structure composed of support structures 5 and connectors 6. The illustrated regions of the main body have a uniform average expandability. All the elements have substantially the same geometry, strut width and mesh width, so that the main body of this stent exhibits a radially symmetrical force distribution during the expansion of the stent.

is FIG. 1B shows part of a first embodiment of a stent according to the invention, which has a radially asymmetrically expandable portion 2. The radially asymmetrically expandable portion 2 is characterized in that the average strut width of the arched elements of the support structures is reduced in comparison to the average strut with of the arched elements of the support structures adjacent to the radially asymmetrically expandable portion. As a result, the expandability is increased and this allows a preferential expansion in the portion 2 during the expansion of the stent. In order to further increase the effect on the expandability, the average strut width not only of the arched elements but additionally also the average strut width of the diagonal elements and/or of the connectors may be reduced in comparison to the average strut width of the corresponding structures in the rest of the main body.

FIG. 1C shows part of a second embodiment of a stent according to the invention, which has a radially asymmetrically expandable portion 3. The radially asymmetrically expandable portion 3 is characterized in that use is made of connectors having a geometry which differs from the geometry of the connectors in the other regions of the main body surrounding the portion 3, so that the radially asymmetrically expandable portion 3 has an increased expandability in comparison to the expandability of the other regions of the main body surrounding the portion 3.

FIG. 1D shows part of a third embodiment of a stent according to the invention, which has a radially asymmetrically expandable portion 4. The radially asymmetrically expandable portion 4 is characterized in that, in the radially asymmetrically expandable portion 4, the spacing between the successive support structures is increased by using connectors which are longer than the connectors in the other regions of the main body surrounding the portion 4. As a result, the main body has an increased expandability in the portion 4 compared to the expandability of the other regions of the main body surrounding the portion 4.

### Exemplary Embodiment 2

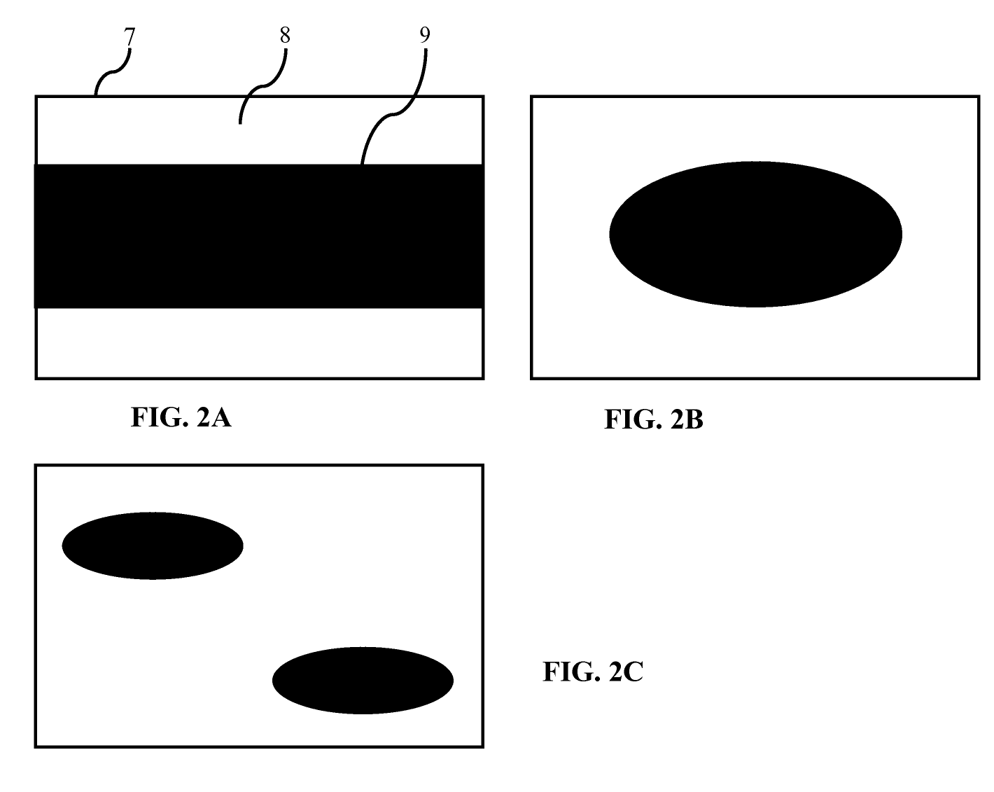

FIGS. 2A-C schematically show selected configurations of radially asymmetrically dilatable portions of a stent according to the invention. FIG. 2A shows part of the main body 7 of a stent according to the invention with a radially asymmetrically dilatable portion 9 in the shape of a strip. Here, the radially asymmetrically dilatable portion 9 extends along the longitudinal axis of the part of the main body 7. The radially asymmetrically dilatable portion 9 is delimited laterally by a region 8 which has a lower expandability than the radially asymmetrically dilatable portion 9.

FIG. 2B schematically shows a radially asymmetrically dilatable portion which is in the shape of an ellipse.

FIG. 2C shows a part of a main body which has two radially asymmetrically dilatable portions, wherein in this case both radially asymmetrically dilatable portions are shown in the shape of an ellipse by way of example. The radially asymmetrically dilatable portions may of course also assume different shapes.

Exemplary Embodiment 3

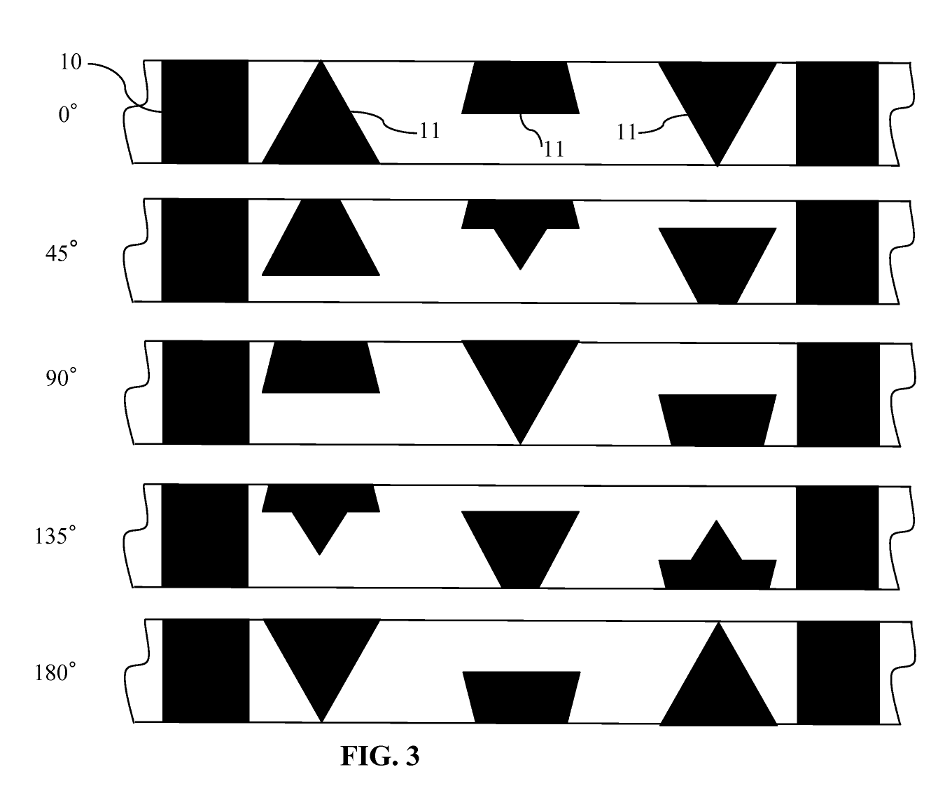

FIG. 3 schematically shows an arrangement of markers on the surface of a catheter according to the invention for applying a stent according to the invention, the markers being positioned and arranged in such a way that the rotational position of the catheter in the vessel can be clearly determined even in a two-dimensional display (as a “plan view”). The catheter has on its surface markers in the form of two strips 10 and at least one triangle 11, preferably two triangles 11, particularly preferably three triangles 11.

Here, the three triangles 11 are oriented and adapted relative to one another in such a way that the rotational position of the catheter in the vessel can be determined in a clear and precise manner from the two-dimensional image thereof. If the rotational position of the catheter is known, the position of the radially asymmetrically expandable portion of the stent according to the invention that is crimped onto the catheter can also be determined. To this end, the three triangles 11 are arranged in an alternating manner in terms of the orientation of the tips. The triangles 11 extend in terms of their size from the tip to the base substantially in each case around half the circumference of the catheter. As shown in FIG. 3, this arrangement of the triangles 11 results in a unique two-dimensional image for each rotational position (0° to 180°), so that the rotational position can be determined on the basis of obtained two-dimensional images. It is sufficient if at least one triangle 11 can be imaged. However, in order to minimize the effect of artifact formation during the imaging, it may be useful to use a number of these triangles 11 in a different rotation and orientation relative to one another, it being possible for two triangles to have the same orientation but different rotational positions. Preferably X-ray markers, fluorescent markers and/or combinations thereof are used.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An expandable stent comprising a tubular main body with a lumen along a longitudinal axis, characterized in that the main body has at least one radially asymmetrically expandable portion, wherein the radially asymmetrically expandable portion has an average expandability which is increased in comparison to an average expandability in a rest of the main body of the stent.

2. The stent according to claim 1, characterized in that a ratio δ of the average expandability in the rest of the main body of the stent to the average expandability in the radially asymmetrically expandable portion is 1>δ≧0.01, optionally 0.95≧δ≧0.1 and optionally 0.9≧δ≧0.7.

3. The stent according to claim 1, characterized in that the main body of the stent has a plurality of circumferential support structures which are arranged one after the other along the longitudinal axis and are each composed of a radial sequence of diagonal elements and arched elements; and the main body has one or more connectors, wherein two successive circumferential support structures are connected to one another via at least one connector.

4. The stent according to claim 3, characterized in that the arched elements in the radially asymmetrically expandable portion have an average strut width which is smaller than an average strut width of the arched elements in the rest of the main body.

5. The stent according to claim 4, characterized in that the ratio φ of the average strut width of the arched elements in the radially asymmetrically expandable portion to the average strut width of the arched elements in the rest of the main body of the stent is 1>φ≧0.01, preferably 0.95≧φ≧0.1 and particularly preferably 0.9≧φ≧0.7.

6. The stent according to claim 3, characterized in that an average mesh width in the radially asymmetrically expandable portion is increased in comparison to an average mesh width in the rest of the main body.

7. The stent according to claim 6, characterized in that a ratio κ of the average mesh width in the rest of the main body of the stent to the average mesh width in the radially asymmetrically expandable portion is 1>κ≧0.01, optionally 0.95≧κ≧0.1 and optionally 0.9≧κ≧0.7.

8. The stent according to claim 3, characterized in that an average spacing between the successive circumferential support structures in the radially asymmetrically expandable portion is greater than an average spacing between the successive circumferential support structures in the rest of the main body of the stent.

9. The stent according to claim 8, characterized in that a ratio e of the average spacing between the successive circumferential support structures in the rest of the main body of the stent to the average spacing between the successive circumferential support structures in the radially asymmetrically expandable portion is 1>ε≧0.01, optionally 0.95≧ε≧0.1 and optionally 0.9≧ε≧0.7.

10. A catheter for applying a stent according to claim 1, wherein the stent is arranged on the catheter.

11. The catheter according to claim 10, characterized in that a surface of the catheter has one or more markers which are applied and oriented in such a way that, during positioning of the catheter in a vessel of an individual to be treated, the spatial orientation of the radially asymmetrically dilatable portion of the stent can be determined, optionally the rotational position of the radially asymmetrically dilatable portion of the stent can be determined.

12. The catheter according to claim 11, characterized in that the markers are substances which can be detected and/or displayed by imaging processes and are preferably X-ray and/or fluorescent markers.

13. The catheter according to claim 11, characterized in that the markers are arranged in different or identical geometric shapes on a surface of a catheter shaft.

14. The catheter according to claim 13, characterized in that the geometric shapes comprise rings, strips, triangles and/or arrows.

15. The catheter according to claim 13, characterized in that at least three identical or different geometric shapes are oriented on the surface of the catheter shaft in such a way that the rotational position of the radially asymmetrically dilatable portion(s) can be determined.
